(19) United States
(12) Patent Application Publication
(10) Pub. No.: US 2004/0265840 A1
(43) Pub. Date: Dec. 30, 2004

(54) Preservation of RNA quality and content in tissue sections during immunohistochemistry

(76) Inventors: Steven Kunitake, San Carlos, CA (US); Anne Mennis, Aptos, CA (US); Pradeepa Vishnu, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/647,031

(22) Filed: Aug. 21, 2003

Related U.S. Application Data
(60) Provisional application No. 60/405,497, filed on Aug. 21, 2002.

(51) Int. Cl. C12Q 1/68; G01N 33/53; G01N 33/567

(52) U.S. Cl. 435/6; 435/7.2

(57) ABSTRACT

This invention relates to methods in immunohistochemistry which preserve and/or maintain the quality and content of RNA during immunohistochemical staining. More specifically this invention relates to methods which preserve the quality and content of RNA during immunohistochemical staining for Laser Capture Microdissection of samples followed by analysis of RNA.

### CROSS-REFERENCE TO RELATED APPLICATIONS

This application claims benefit of priority from U.S. Provisional Patent Application 60/405,497, filed Aug. 21, 2002, which is hereby incorporated by reference as if fully set forth.

### TECHNICAL FIELD

This invention is in the field of immunohistochemistry (IHC), specifically the preservation of RNA quality and content in combination with IHC staining. Compositions comprising IHC stained samples wherein the RNA quality and content has been maintained are provided. Methods comprising the use of agents to prevent the degradation and/or loss of RNA during IHC are also provided.

### BACKGROUND ART

Immunohistochemistry (IHC) staining of tissue sections is a powerful tool for identifying cells that express specific proteins. With the advent of Laser Capture Microdissection (LCM), it is now possible to isolate these specific cells and perform additional molecular analyses.

One interesting analysis is the determination of the gene expression profile from mRNA isolated from the IHC stained sample. However, IHC protocols require several aqueous incubations during which RNases, particularly intrinsic RNases found in the tissue or cell sample, can degrade the cellular RNA. Limiting the duration of aqueous incubation can minimize, but not eliminate, the extent of RNA degradation.

The presence of an RNase inhibitor during this process might preserve RNA integrity in the sample, but large molecule RNase inhibitors (anti-RNase antibodies, placental RNase inhibitor, etc.) were found to diffuse too slowly to block intrinsic RNases. Most small molecule RNase inhibitors are ineffective or are incompatible with the IHC process.

The above statements in this section are based on the information available to the applicant and does not constitute any admission as to the correctness of the statements.

### DISCLOSURE OF THE INVENTION

This invention provides methods in immunohistochemistry (IHC) that preserve RNA quality and content in a tissue or cell sample. More specifically, this invention provides methods that preserve RNA quality and content in IHC staining procedures by utilizing a ribonucleoside vanadyl complex (RVC) as an RNase inhibitor. This invention also provides methods which preserve RNA quality and content in IHC staining procedures by performing said procedure at low temperatures in the presence or an RNase inhibitor, optionally for short incubation or treatment periods. The invention also relates to a combination of said methods in IHC staining procedures.

In a first aspect, the invention provides methods in IHC that preserve or maintain RNA quality and content during IHC staining by using a RVC as an RNase inhibitor. The methods may also be used to inhibit (intrinsic or endogenous) RNase activity in an IHC sample.

In a second aspect, the invention provides methods in IHC that preserve or maintain RNA quality and content during IHC staining, said method comprising performing one or more acts required for IHC staining at a temperature below +15° C. or thereabouts, such as, but not limited to +4° C., in the presence of an RNase inhibitor. Non-limiting examples of possible RNase inhibitors include, but are not limited to, a RVC. Again, the methods may be used to inhibit (intrinsic or endogenous) RNase activity in an IHC sample.

In a third aspect, the invention provides methods in IHC that preserve or maintain RNA quality and content during IHC staining, said method comprising performing one or more acts required for IHC staining at a temperature below +15° C. or thereabouts, such as, but not limited to +4° C., in the presence of an RNase inhibitor and for short incubation (or treatment) times. Non-limiting examples of such times are between about 3 minutes to about 1 minute. Possible RNase inhibitors include, but are not limited to, a RVC. Again, the methods may be used to inhibit (intrinsic or endogenous) RNase activity in an IHC sample.

In another aspect, the invention provides methods in IHC that preserve or maintain RNA quality and content in an IHC sample as described above for analysis by laser capture, such as by Laser Capture Microdissection (LCM). Alternatively, the samples may be dissected, or microdissected, by other means. Such IHC samples include, but are not limited to, fixed-frozen samples, fresh samples, and samples that have been formalin fixed and paraffin embedded (FFPE samples).

In a further aspect, the invention provides IHC samples comprising an RNase inhibitor, such as a RVC. Such samples may be used for the analysis or extraction RNA present in the sample. Non-limiting examples of such analysis or extraction include in situ detection or quantitation of RNA in the sample, extraction of total RNA from the sample for further analysis, and microdissection of the sample for isolation of a portion thereof from which RNA may be extracted and analyzed.

### MODES OF CARRYING OUT THE INVENTION

This invention provides methods in immunohistochemistry (IHC) that preserve or maintain RNA quality and content. Stated differently, the invention provides for the prevention of RNA degradation, destruction and/or loss from a sample undergoing, or after, IHC staining. Immunohistochemistry may be considered generally as the detection of one or more subcellular or extracellular components in a cell containing tissue sample or section by use of detectable markers. The detection is normally mediated by the use of a (primary) antibody that specifically binds a subcellular component. The component may thus be considered the antigen in a specific antibody-antigen binding reaction. The antibody may be linked to a detectable marker (such as, but not limited to, biotin) or may itself be recognized by a detectable reagent (such as a secondary antibody or streptavidin) which binds the antibody. Fluorescent dyes or enzymes such as, for example, horseradish peroxidase are often used to make the reagent detectable.

In one embodiment of the invention, the invention provides methods that preserve or maintain RNA quality and content in an IHC sample, such as for LCM, by use of a ribonucleoside vanadyl complex (RVC) as an RNase inhibitor. RVCs unexpectedly provide superior inhibition of RNase activity in a processed tissue when compared to other commercially available RNase inhibitors. Use of RVCs is preferred in the practice of the invention over other RNase inhibitors and allows for performance of IHC staining of samples for LCM and downstream gene expression analysis.

RVCs may be obtained by methods known in the art or are obtained commercially. RVCs are RNase inhibitors, but have not been used in combination with IHC. Prior to the instant invention, the successful application of RVCs in combination with the protocols used for IHC staining was unknown. RVCs have been used in solution to stabilize RNA during RNA isolation from resting lymphocytes (Berger et al. Biochemistry 1979, 18:51431) in which the compound was used throughout the procedure of cell fractionation. It has also been used during cDNA production and to protect RNA during digestion of DNA by DNase (Pukas et al., Biochemistry 1982, 21:46023) and during all washing steps in an in situ hybridization procedure to stabilize mRNA by inhibiting endogenous RNases (Kanz et al. Exp. Hematol. 1988, 16:394).

As used in the practice of the invention, RVCs may be used at various concentrations, such as, but not limited to, about 5, about 10, about 15, about 20, and about 25 mM as well as ranges therein from about 5 to about 25 mM. IHC samples that have been treated with such concentrations of RVC, and thus retain the RVC in the sample, are within the scope of the invention.

Another embodiment of the invention provides methods that preserve or maintain RNA quality and content in IHC staining procedures, such as for LCM, by use of reduced temperatures in combination with an RNase inhibitor during IHC. Exemplary temperatures include any below room temperature, preferably below about +15° C., below about +12° C., below about +10° C., below about +8° C., below about +6° C., or about +4° C. Performance of IHC staining below +15° C., such as at +4° C., significantly increases the yield and quality of retrieved RNA as measured by analysis of 18S and 28S ribosomal RNA and real time quantitative RT-PCR. The combination of reduced temperature IHC staining and use of an RNase inhibitor is optionally performed in a manner such that one or more incubation times in the IHC protocol is for a reduced time, such as less than or about 2 hours, less than or about 90 minutes, less than or about 60 minutes, less than or about 30 minutes, less than or about 15 minutes, less than or about 10, or less than or about 5 minutes. Non-limiting examples include between about 3 minutes and about 1 minute. In preferred embodiments of the invention, various times during an IHC staining process are as follows: 3 minutes or thereabouts for the primary antibody and 1 minute or thereabouts for the secondary antibody (or reagent that binds the primary antibody). In a particularly preferred embodiment, the above times are used in combination with the necessary washes (exemplified in the examples below) to result in a total time for the overall process of about 5 to about 10 minutes. In other embodiments of the invention, the total time may be up to about 2 hours.

The use of reduced incubation times may be with respect to any act during IHC, but is preferably with respect to the contacting of a sample with a primary antibody (optionally labeled or conjugated with another moiety such as biotin) and/or the subsequent contacting of a sample with a secondary antibody (optionally labeled) or a reagent that binds said primary antibody.

The methods of the invention are applicable to a wide range of samples and tissue sections, including those of normal tissues or cells or abnormal/non-normal tissues or cells such as those associated with cancer. Cells from a culture may also be used as a sample. Non-limiting examples of cells for use in the practice of the present invention include, but are not limited to, primary cells, cultured cells, tumor cells, non-tumor cells, blood cells, cells of the pituitary or other endocrine glands, bone cells, lymph node cells, brain cells, lung cells, heart cells, spleen cells, breast cells, prostate cells, colon cells, skin cells, ovary cells, uterine cells, liver cells, kidney cells, and vascular tissue cells.

The present invention may also be applied to tissues (and cell types therein) involved in, or associated with, any disease or undesired condition. For example, and without limiting the invention, the present invention may be used with neuronal and non-neuronal cells involved in disorders of the nervous system, such as, but not limited to, neurodegenerative diseases, including Parkinson's disease and Alzheimer's disease; multiple sclerosis; and psychiatric disorders. Similarly, the invention may be practiced with non-neuronal cells associated with such disorders (including, but not limited to microglial cells, astrocytes, oligodendricytes, and infiltrating inflammatory cells).

The invention may also be practiced with cells associated with disorders of the cardiovascular and urinary systems. Examples from the area of cardiovascular disease include, but are not limited to, smooth muscle cells, endothelial cells and macrophages while examples from kidney disorders include, but are not limited to, cells of the cortex, medulla, glomerulus, proximal and distal tubules, Bowman's capsule and the Loop of Henley.

Inflammatory and autoimmune diseases are additional non-limiting examples of disorders wherein the tissues and cells involved in or associated therewith may be used in combination with the present invention. Examples of such disorders include rheumatoid arthritis, myasthenia gravis, lupus erythematosus, certain types of anemia, multiple sclerosis, and juvenile-onset diabetes. Cells involved in such diseases include neutrophils, eosinophils, basophils, monocytes, macrophages, lymphocytes,

Non-limiting examples of cancer cells include those from sarcomas, carcinomas, lymphomas, leukemias, breast cancer, prostate cancer, lung cancer, colorectal cancer, soft tissue cancers, biopsies, skin cancer, brain cancer, liver cancer, and ovarian cancer. Preferably, the tissues or cells used in the practice of the invention are from a human subject.

Methods for fixing and/or embedding tissues or cells are known in the art (Advanced Laboratory Methods in Histologv and Pathology, (U.V. Mikel, Ed.) Armed Forces Institute of Pathology, American Registry of Pathology, Washington, D.C. 1994: Methods in Molecular Biology: Immunocytochemical Methods and Protocols, Vol. 34 (L. C. Javois, Ed.) Humana Press, Totowa, N.J. 1994; Immunocytochemical Techniques: Principles and Practice, Beltz and Burd, Blackwell Scientific Publications, Inc., Cambridge, Mass. 1989).

For the practice of the invention, any IHC protocol known, or which may come within known or customary practice within the art, may be used in combination with the improvements and features as described herein. Therefore, the present invention may also be considered an improvement in methods of IHC wherein the improvement comprises the use of an RNase inhibitor, reduced temperatures, and/or reduced incubation times as described herein.

The use of an RNase inhibitor as described herein is preferably with respect to all acts used in the handling or treatment of a sample prior to, during, and after IHC. The use of reduced temperature and/or reduced incubation times is with respect to one or more of the acts used in the handling or treatment of a sample prior to, during, and after IHC.

Also provided by the invention are kits for use in the practice of the methods disclosed herein, where such kits may comprise containers, each with one or more of the various reagents (typically in concentrated form) utilized in the methods, including, for example, buffers and the appropriate IHC reagents of the present invention. A label or indicator describing, or a set of instructions for use of, kit components in an IHC method of the present invention, will also be typically included, where the instructions may be associated with a package insert and/or the packaging of the kit or the components thereof.

Citation of the documents herein is not intended as an admission that any of the foregoing is pertinent prior art. All statements as to the date or representation as to the contents of these documents is based on the information available to the applicant and does not constitute any admission as to the correctness of the dates or contents of these documents.

The following examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### EXAMPLE 1

### Use of Ribonucleoside Vanadyl Complexes (RVCs) in IHC

The immunostaining was performed on frozen, acetone fixed, sections. RVCs (Ribonucleoside Vanadyl Complexes, Sigma catalog #R3380; Berger et al. Biochemistry 1979, 18:51431; Pukas et al., Biochemistry 1982, 21:46023) at 20 mM were added to all the acts of the staining including primary antibody solution, secondary antibody solution, and rinsing solutions (phosphate buffered saline or PBS). The processing was as follows:

Thaw slides for 30 seconds

Fix in +4° C. acetone for 2 minutes

Rehydrate in PBS with RVC

Apply primary antibody with RVC for 3 minutes

Rinse in PBS with RVC

Apply secondary, fluorophore conjugated antibody (or other detectable reagent that binds the primary antibody) with RVC for 3 minutes

Rinse in PBS with RVC

Dehydrate

Optionally visualize based on immunostaining followed by microdissection

### EXAMPLE 2

### IHC Staining at +4° C.

Generally, IHC staining procedures performed at +4° C. involve prolonged to overnight incubations with primary antibodies to enhance staining, decrease the background staining, and reduce cost of the procedure (Theory and Practice of Histological Techniques. Ed. J. D. Bancroft & A. Stevens, Churchill Livinstone). Such extended times tend to exacerbate the possibility for RNA degradation. Short incubation times at temperatures below about +15° C. have not been used in IHC procedures with the purpose of retrieving high quality RNA from the stained samples.

IHC staining was performed on frozen, acetone fixed sections. All reagent solutions were prepared with nuclease free PBS containing a RVC RNase inhibitor and kept at +4° C. The slides were removed from −80° C. storage, 30 seconds are allowed for the condensation to disappear prior to fixing. The staining procedure was done at +4° C. by keeping the slides on a cold block through all parts of the procedure:

1) fix in acetone (+4° C.) for 2 minutes; air dry for 30 seconds; use hydrophobic barrier pen to circumscribe each section;

2) place the slides on a cold block (+4° C.);

3) apply about 200 μl PBS (with RVC)/section;

4) drain off; place the slides on the cold block and apply about 50 μl/section of the primary biotinylated antibody (with RVC) for 3 minutes;

5) rinse by applying about 200 μl PBS (with RVC)/section; drain and repeat;

6) drain off; place slides on the cold block and apply about 50 μl/section of a Cy3 conjugated streptavidin solution (with RVC) for 1 minute;

7) rinse by applying about 200 μl PBS (with RVC)/section; drain and repeat.

dehydrate at room temperature

8) place in 75% ethanol for 30 seconds;

9) place in 95% ethanol for 30 seconds;

10) place in 100% ethanol (freshly dispensed) for 30 seconds;

11) place in xylene for 5 minutes;

12) dry the slides in a fume hood.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. An immunohistochemically stained tissue or cell sample comprising a ribonucleoside vanadyl complex (RVC) RNase inhibitor applied during said immunohistochemical staining.

2. A method of inhibiting RNAse activity during immunohistochemical staining of a sample, said method comprising the presence of an RNase inhibitor.

3. The method of claim 2 wherein said inhibitor is a ribonucleoside vanadyl complex (RVC).

4. The method of claim 2 wherein said staining is conducted at temperatures below about +15° C.

5. The method of claim 4 wherein said temperature is +4° C.

6. The method of claim 2 wherein said staining comprises contacting a primary antibody solution with said sample for less than 5 minutes.

7. The method of claim 6 wherein said contacting is for about 3 minutes.

8. The method of claim 2 wherein said staining comprises contacting a primary antibody solution with said sample and further comprises contacting said sample with a reagent that binds said primary antibody for less than 5 minutes.

9. The method of claim 8 wherein said contacting with a reagent that binds said primary antibody is for about 3 minutes.

10. A method of immunohistochemically staining a tissue or cell sample comprising the use of an RNase inhibitor during said staining.

11. The method of claim 10 wherein said inhibitor is a ribonucleoside vanadyl complex (RVC).

12. The method of claim 10 wherein said staining is conducted at temperatures below about +15° C.

13. The method of claim 12 wherein said temperature is +4° C.

14. The method of claim 10 wherein said staining comprises contacting a primary antibody solution with said sample for less than 5 minutes.

15. The method of claim 14 wherein said contacting is for about 3 minutes.

16. The method of claim 10 wherein said staining comprises contacting a primary antibody solution with said sample and further comprises contacting said sample with a reagent that binds said primary antibody for less than 5 minutes.

17. The method of claim 16 wherein said contacting with a reagent that binds said primary antibody is for about 3 minutes.

18. An improved immunohistochemistry staining method, said improvement comprising the use of an RNase inhibitor.

19. The method of claim 18 wherein said inhibitor is a ribonucleoside vanadyl complex.

20. The method of claim 18 further comprising an improvement selected from the use of temperatures below about +15° C. and the use of reduced incubation times.
